(19) United States
(12) Patent Application Publication
(10) Pub. No.: US 2014/0276054 A1
(43) Pub. Date: Sep. 18, 2014

(54) Piezoelectric-Actuated Fluid-Delivery Devices and Associated Systems and Methods

(71) Applicant: Volcano Corporation, San Diego, CA (US)

(72) Inventors: Norman H. Hossack, Folsomm, CA (US); Stephen Davies, El Dorado Hills, CA (US)

(73) Assignee: Volcano Corporation, San Diego, CA (US)

(21) Appl. No.: 14/213,050

(22) Filed: Mar. 14, 2014

Related U.S. Application Data
(60) Provisional application No. 61/794,002, filed on Mar. 15, 2013.

(51) Int. Cl. A61M 5/145; A61B 8/12

(52) U.S. Cl. CPC A61M 5/145; A61B 8/12
USPC 600/439; 604/131; 604/500

(57) ABSTRACT

Minimally invasive therapeutic fluid delivery devices, systems, and methods are disclosed. In some embodiments, the devices include at least a flexible elongate member with a chamber situated in the distal portion thereof, the chamber having an inlet and an outlet and a piezoelectric actuator that alters a volume of the chamber to eject a portion of the therapeutic fluid. In one embodiment, the device includes an array of piezoelectrically actuatable chambers that can eject a droplet of a drug orthogonally to the length of the flexible elongate member. In another embodiment, the device includes a piezoelectrically actuatable chamber that ejects along the length of the flexible elongate member.

### CROSS-REFERENCE TO RELATED APPLICATIONS

The present application claims priority to and the benefit of U.S. Provisional Patent Application No. 61/794,002, filed Mar. 15, 2013, which is hereby incorporated by reference in its entirety.

### TECHNICAL FIELD

The present disclosure relates to elongated tubular devices, systems, and methods utilized in minimally invasive delivery of therapeutic fluids to internal cites within a patient. In particular, the present disclosure relates to targeted fluid delivery devices, systems, and methods that administer therapeutic fluids utilizing piezoelectrically actuated dispensing mechanisms.

### BACKGROUND

Many therapeutic compounds have been developed to treat numerous conditions within the body. Unfortunately, many of these compounds are delivered orally or by injections such that the entire body experiences the effects of the compounds in a systemic manner. Efforts have been made to deliver smaller amounts of the compounds closer to the site of treatment. For instance, a drug-coated device may be inserted into a patient, relying on elution of the drug off the surface of the device for delivery. However, measuring the amount of drug dispensed in such a situation can be very difficult and it may not be possible to get the drug carrying stent as close to the diseased tissue as necessary. Alternatively, a needle may be used at the end of a minimally invasive catheter delivery system. For remote locations within the body, such as within the heart, blood vessels or organs, the size of the delivery system must be very small and flexible to gain access to the area needing treatment. In certain designs, as the needle attempts to penetrate the tissue, the catheter pushes away from the vessel wall making it almost impossible to insert the needle in the desired location. One approach to solve this problem has been to add hooks on the end of the delivery system to penetrate the vessel walls to maintain the position of the delivery catheter while the needle is advanced. These hooks, as well as the needle itself, cause trauma to the vessel walls. In addition, the bore of the needle may be large compared with the amount of the drug to be administered. Thus, measuring the precise amount of the drug dispensed is problematic.

Therefore, while there have been some significant improvements in targeted drug delivery, the existing fluid delivery systems have not been entirely satisfactory.

### SUMMARY

Embodiments of the present disclosure are directed to minimally invasive therapeutic fluid delivery devices, systems, and methods.

In some embodiments, a flexible therapeutic fluid delivery device is provided. The delivery device includes a flexible elongate member having a proximal portion and a distal portion. The flexible elongate member is configured for minimally invasive insertion into a patient. The delivery device further includes a chamber situated in the distal portion of the flexible elongate member, a piezoelectric actuator positioned adjacent to the chamber, and a plurality of electrical leads coupled to the piezoelectric actuator. In the delivery device, the chamber has an outlet configured to eject a therapeutic fluid, and a piezoelectric actuator is positioned adjacent to the chamber such that, when actuated, the piezoelectric actuator alters a volume of the chamber to eject a portion of the therapeutic fluid.

In some embodiments, a flexible therapeutic fluid delivery device is provided. The delivery device includes a flexible elongate member having a proximal portion and a distal portion, with a cylindrical section in the distal portion. The cylindrical section includes an array of chambers formed in a substrate, each of the chambers having an outlet permitting the ejection of a portion of a therapeutic fluid filling the chamber and an inlet. A portion of the substrate has electrical conductors. The cylindrical section further includes an array of piezoelectric actuators coupled to the electrical conductors, each piezoelectric actuator coupled to one of the array of chambers such that, when actuated, the piezoelectric actuator ejects the portion of the therapeutic fluid and a reservoir, containing therapeutic fluid, situated in the distal portion of the flexible elongate member and coupled to the inlet of each of the array of chambers.

In some embodiments, a method for applying a therapeutic fluid to a tissue within a patient using an elongated fluid delivery device is provided. The method includes steps of inserting the elongated fluid delivery device into a patient, the elongated fluid delivery device having an electrically actuated ejection system adjacent a distal end thereof and of positioning the distal end of the elongated fluid delivery device close to the tissue. The method further includes steps of actuating the ejection system to eject a portion of a therapeutic fluid with sufficient velocity to contact the tissue and of removing the elongated fluid delivery device from the patient.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a diagram of a steerable intravascular device having a fluid delivery component at a distal end according to an embodiment of the present disclosure.

FIGS. 2A and 2B are partial cross-sectional perspective views of a fluid delivery tip of FIG. 1 in two different states according to an embodiment of the present disclosure.

FIG. 3 is a diagrammatic perspective view of a piezoelectric actuator in two different states of activation.

FIGS. 4A, 4B, and 4C illustrate cross-sectional side views of fluid delivery tips in various states of activation according to embodiments of the present disclosure.

FIGS. 5A and 5B are cross-sectional views of a fluid delivery chamber in two states of activation according to an embodiment of the present disclosure.

FIG. 6 is a cross-sectional view of an array of piezoelectrically actuated chambers according to an embodiment of the present disclosure.

FIG. 7 is a perspective diagram of an array of piezoelectrically actuated chambers formed into a treatment device prior to incorporation into an intravascular device according to an embodiment.

FIG. 8 is an axial cross-sectional diagram of an array of piezoelectrically actuated chambers according to an embodiment of the present disclosure.

FIG. 9 is a longitudinal cross-sectional diagram of an array of piezoelectrically actuated chambers of FIG. 8.

FIG. 10 is a diagram of a drug delivering intravascular device within a vessel according to an embodiment of the present disclosure.

FIG. 11 is a diagram of a drug delivering intravascular device within a vessel according to an additional embodiment of the present disclosure.

FIG. 12 is a flowchart of a method for applying a therapeutic fluid to a tissue within a patient using an intravascular device according to an embodiment of the present disclosure.

For clarity of discussion, elements having the same designation in the drawings may have the same or similar functions. The drawings may be better understood by referring to the following Detailed Description.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

As used herein, “flexible elongate member” or “elongate flexible member” includes at least any thin, long, flexible structure that can be inserted into the tubular passages, including arteries and veins, of a patient. While the illustrated embodiments of the “flexible elongate members” of the present disclosure have a cylindrical profile with a circular cross-sectional profile that defines an outer diameter of the flexible elongate member, in other instances all or a portion of the flexible elongate members may have other geometric cross-sectional profiles (e.g., oval, rectangular, square, elliptical, etc.) or non-geometric cross-sectional profiles. Flexible elongate members include, for example, guidewires and catheters. In that regard, catheters may or may not include a lumen extending along its length for receiving and/or guiding other instruments. If the catheter includes a lumen, the lumen may be centered or offset with respect to the cross-sectional profile of the device.

“Connected”, “coupled”, and variations thereof as used herein includes direct connections, such as being glued or otherwise fastened directly to, on, within, etc. another element, as well as indirect connections where one or more elements are disposed between the connected elements.

“Intravascular device”, as used herein includes hypotubes, catheters, guidewires, and other devices such as may be used by a doctor to provide various treatments, and to obtain data from vessels throughout the body.

“Therapeutic fluid” is intended to be a fluid, including emulsions containing particles, that may provide a therapeutic effect for a patient, such as but without limitation, drugs, biologics, cells, such as stem cells, enzymes, and chemical compounds. While many embodiments are described herein in relation to drug delivery, any therapeutic fluid can be delivered in a similar manner.

Referring now to FIG. 1, therein is a schematic depiction of a steerable intravascular device 100 having certain drug delivery capabilities. As depicted, the steerable intravascular device 100 includes a flexible elongate member 102 coupled at a proximal portion 106 to a controller 104 that allows a user to steer a distal portion 108 of the flexible elongate member 102. Catheter steering assemblies are well known and the steering assembly will not be further discussed herein. The flexible elongate member 102 is generally flexible and is formed from a biocompatible polymer or metal or a material having a bio-compatible coating thereon. Additionally, the flexible elongate member 102 may have one or more lumens extending therethrough and may have a length ranging from about 0.3 meters to about 2 meters and a diameter ranging from about 0.3 millimeters to about 5 millimeters.

The controller 104 may be configured to supply power and/or electrical communication signals to various components in embodiments of steerable intravascular device 100. A fluid delivery component 110 is situated within a lumen within the distal portion 108 of the flexible elongate member 102 and provides small portions or doses of a therapeutic fluid to a site within a patient close to the distal portion 108 of flexible elongate member 102. The distal portion 108 may be steered using the controller 104 to bring the fluid delivery component 110 into a desired position. The controller 104 may also be used to activate the fluid delivery component 110 to eject a portion of the therapeutic fluid. In some embodiments, the fluid delivery component 110 is configured entirely within the flexible elongate member 102; while in other embodiments, a portion of the fluid delivery component 110 may protrude beyond a distal end of the flexible elongate member 102. In some embodiments the therapeutic fluid is a conventional pharmaceutical, chemical compounds, or a biologic; while in other embodiments the therapeutic fluid includes cells, such as stem cells, or other bio-entities. When the patient is suffering from a malady, a physician may carefully guide the fluid delivery component 110 to a site within the patient requiring treatment and dispense an amount at required. The device may further include an imaging component 120 such as an ultrasound transducer, disposed adjacent to the distal end 108. The imaging component 120 aids the physician in targeting the tissue for treatment.

Referring now to FIGS. 2A and 2B, therein is provided further detail regarding the fluid delivery component 110 and the distal portion 108 of flexible elongate member 102. FIG. 2A provides an illustration in which a fluid delivery component 110 is in an on or actuated state, while FIG. 2B depicts the fluid delivery component 110 in an “off” or dormant state. In the depicted embodiment, the fluid delivery component 110 includes a piezoelectric actuator that has two piezoelectric actuator components. Thus, as depicted the piezoelectric actuator portion of the fluid delivery component 110 includes a top actuator component 202 and a bottom actuator component 204. In the depicted embodiment, each of the piezoelectric actuator components 202 and 204 is formed from two piezoelectric elements that are paired together for improved performance. As labeled in FIG. 2A, the top actuator component 202 includes an expanding piezoelectric element 206A depicted on top, and a contracting piezoelectric element 206 B depicted on the bottom. It will be appreciated that by layering the piezoelectric elements in this manner, greater force can be generated.

As mentioned, FIG. 2A depicts the fluid delivery component 110 in an on or activated state. This state is achieved when an activation energy is supplied by electrical leads 208. The activation energy deforms the piezoelectric actuator components 202 and 204, increasing a volume therebetween. The volume is referred to as a chamber formed by the piezoelectric actuator components 202 and 204 and by retaining walls on the side. In one aspect, a flexible polymer extends between the piezoelectric actuator components 202 and 204 to form flexible sidewalls. In an alternative form, the tolerance between the retaining side walls and the actuator components 202 and 204 is sufficient tight to retain fluid within the chamber. The chamber is filled at least partially with the therapeutic fluid in both the on and “off” states. As the volume between actuator components 202 and 204 increases, additional fluid is drawn into the expanded chamber from a coupled reservoir 210. When the activation energy supplied by the electrical leads 208 is withdrawn or ceases to be supplied the actuator components 202 and 204 return to their dormant state and to the corresponding dormant shape. In so doing, the expanded chamber contracts causing a sudden increase of pressure therein. The increased pressure also closes a check valve (not shown) on the line to a reservoir 210 that prevents a back flow of fluid from the chamber into the reservoir 210. The rapid decrease in the volume of the chamber forces out a portion 212 of the therapeutic fluid in a focused jet, stream, or one or more droplets of fluid. In the depicted embodiment, the portion of therapeutic fluid exits the chamber and the distal portion 108 of flexible elongate member 102 through a conduit 214.

FIG. 3 is a diagram providing further detail regarding the activation of actuator components 202 and 204. As discussed above, each of actuator components 202 and 204 includes two piezoelectric elements that have opposite reactions to an activation energy. The piezoelectric elements are formed from lead zirconate titanate, PZT, but materials such as barium titanate and others may also be used. In FIG. 3, actuator component 202 is depicted in an “off” state on top and an “on” state below. Actuator component 202 includes a piezoelectric element 206A that when exposed to an activation energy elongates or expands. Meanwhile, when exposed to the same activation energy the piezoelectric element 206B contracts, decreasing in size. When piezoelectric elements 206A and 206 B are combined to form an actuator component 202, rather than simply increasing or decreasing in length, the actuator component 202 bends. This bending motion provides a change in volume of the chamber that lasts only while the activation energy is provided. Once the activation energy is removed the piezoelectric elements 206A and 206B return to an “off” state.

As will be disclosed below, some embodiments of fluid delivery component 110 do not include paired piezoelectric elements, but instead include a single piezoelectric element that is constrained in such a manner that activation of the piezoelectric element causes the volume of the chamber to change in such a way that a force is applied to the therapeutic fluid contained in the volume.

FIG. 4A-C also provides further detail regarding the actuation of actuator components 202 and 204 in an embodiment of a fluid delivery component 110. FIG. 4A is a cross-sectional diagram of a fluid delivery component 110 such as may be configured within the distal portion or distal tip of a flexible elongate member of an intravascular device, such as that of intravascular device 100 of FIG. 1. In the “on” state diagram of FIG. 4A both actuator components 202 and 204 have an activation energy applied thereto, causing the actuator components to bend (component 202 bends upward and component 204 bends downward), expanding the volume of fluid filled chamber 402.

A distal end of the chamber includes a tapered outlet or nozzle 404. The tapering may serve to better channel the force exerted by the actuator components 202 and 204 as they return to their dormant state and shape, thereby creating a focused jet or stream of fluid exiting the nozzle 404. The fluid delivery component 110 also includes a reservoir connector 406 that allows the chamber 402 to be in fluid communication with a reservoir containing a larger amount of the therapeutic fluid. As the chamber 402 expands when subjected to an activation energy, fluid flows from the reservoir through the reservoir connector 406 through a check valve 408 and into the chamber 402. As may be seen in FIGS. 4B and 4C, when the activation energy is removed and the chamber 402 forcefully decreases in size the check valve 408 closes. This prevents therapeutic fluid from flowing out of chamber 402 back through the reservoir connector 406 and into the reservoir, thus forcing the therapeutic fluid through the tapered outlet 404. In an alternative form, the inline check valve may be replaced by side wall ports that will be exposed at the top of the actuator stroke shown in FIG. 4A such that fluid may fill the chamber. These ports will be covered as the actuators move to the collapsed position of FIG. 4B thereby closing the ports and causing compressed fluid to exit the nozzle.

The expelled portion of therapeutic fluid is a short stream or droplet 412 expelled with adequate force to bring it into contact with a targeted tissue near the distal portion 108 of flexible elongate member 102. Droplet 412 may contain a volume of less than about a microliter. In some embodiments, the droplet 412 has a volume from about 5 to about 25 picoliters In general, the volume of droplet 412 may range in size from one or more picoliters to one or more microliters. The velocity at which droplet 412 is ejected from the chamber 402 may vary according to the dimensions of chamber 402 and with the amount of activation energy supplied. For example, droplet 412 may be expelled at a velocity of about 10 to about 25 meters per second. In some embodiments, the velocity may be even greater by changing the return force of the piezoelectric actuator components 202 and 204 and/or the shapes of nozzle 404 and chamber 402. Droplet 412 may be expelled with sufficient force to cause it to penetrate a depth into the target tissue. The velocity of the expelled droplet 412 is related to the activation energy applied to the fluid delivery component 110 such that a larger activation energy produces a higher velocity. A higher velocity may, in turn, cause the droplet 412 to penetrate deeper into the target tissue.

The embodiment of fluid delivery component 110 depicted in FIG. 4C is substantially similar to the embodiment depicted in FIG. 4B. The embodiment of FIG. 4C further includes a straight section 414 coupled to the tapered outlet 404. The straight section 414 may extend up to or beyond a housing in which the fluid delivery component 110 is positioned in the distal portion 108 a flexible elongate member 102 of intravascular device 100. In some embodiments, the straight section 414 may be formed from a different material than the tapered outlet 404 or may be formed from different pieces of the same material. The straight section 414 and the tapered outlet 404 may be coupled adhesively or by press-fitting.

FIGS. 5A and 5B depict another embodiment of a fluid delivery component or portion thereof. Fluid delivery component 500 includes a single actuator component 502. In some embodiments, the actuator component 502 may have a single piezoelectric element that causes a change in volume by an increase in thickness when subjected to an activation energy and/or by an associated contraction of the walls of an associated chamber. However, the depicted embodiment of actuator component 502 includes a pair of piezoelectric elements 504A and 504B depicted in FIG. 5A in a dormant state and in FIG. 5B in an activated state. In such embodiments, the actuator component 502 may function substantially as depicted in FIG. 3 and as described in the corresponding sections above. The actuator component 502 rests upon a top surface of chamber walls 506, in some embodiments forming a top surface of a chamber 508. The bottom of chamber 508 is provided by a substrate 510 that has a hole therethrough to provide an outlet 512. In the depicted embodiment, an outlet 512 is a tapered outlet forming a nozzle, but in other embodiments it may be have a substantially uniform diameter. An inlet 514 couples the chamber 508 to a reservoir containing a larger volume of therapeutic fluid. To prevent back flow from chamber 508 into the reservoir, a check valve 516 is provided in the inlet 514. FIG. 5 also depicts two electrical leads, including a lead 518A and a lead 518B, that are coupled to a controller. Activation energy is supplied by the controller through electrical leads 518A and 518B.

As depicted in FIG. 5B, when a coupled controller supplies the activation energy through electrical leads 518A and 518B, piezoelectric element 504B expands and piezoelectric element 504A contracts, thereby bending actuator component 502, and decreasing the volume of chamber 508. The bend in the actuator component 502 ejects a droplet 520 made up of a portion of the therapeutic fluid contained in chamber 508. In some embodiments, the piezoelectric elements 504B and 504A are reversed, such that the actuator component bends outward, expanding the chamber 508 which draws in additional therapeutic fluid from a coupled reservoir. Then, when the activation energy is removed actuator component 502 returns to its dormant shape, rapidly decreasing the volume, and thereby ejecting a droplet 520 of the therapeutic fluid.

Where the activation energy is lower, droplet 520 may be ejected with sufficient velocity to contact the target tissue nearby the fluid delivery component 500. When the activation energy is higher, the droplet 520 may be ejected with sufficient velocity to penetrate a certain depth into the target tissue thereby delivering a small, controlled amount of a drug, stem cells, or another therapeutic fluid into the tissue without a needle. Avoiding the use of a needle may facilitate recovery and help limit damage to the tissue being treated. Also, given the speed of the ejection, and the difference in mass associated with the droplet 520 and a catheter containing the fluid delivery component 500, the catheter “floating” within a blood vessel or a cardiac chamber will remain substantially stationary. This may obviate a need of including stabilizing features to hold the catheter close to the tissue of interest.

FIG. 6 is a cross-sectional diagram of an array 600 that includes a plurality of fluid delivery components like fluid delivery component 500, with each of the array having a chamber 508 and an actuator component 502. In order to form the array 600, substrate 510 may be patterned by an etching or milling process. In the depicted embodiment, two separate substrates are patterned to form substrate 510 with a plurality of openings 512 therethrough (the first substrate) and to form chamber walls 506 with a plurality of chambers therein (the second substrate). In other embodiments, both the substrate 510 and chamber walls 506 are formed from a monolithic substrate or structure. During the fabrication process, chambers 508 may be formed as holes extending through the second substrate. When the second substrate is coupled to substrate 510, substrate 510 forms the bottom of the chambers 508 with the second substrate forming the chamber walls 506 of each of chambers 508. In the depicted embodiment, substrate 510 and chamber walls 506 are both made from polyimide. In general, the array 600 is formed from a flexible, biocompatible material. The inlets 514 and check valves 516 may be formed in a top surface of the second substrate by the same etching or milling process used to create chambers 508.

A set of electrical leads 518A may be formed on a top surface of the second substrate prior to or after the etching or milling process used to create chambers 508. A first piezoelectric material layer may be formed over or positioned over the additional substrate (used to form the piezoelectric elements 504B), followed by a second piezoelectric material layer in some embodiments (used to form the piezoelectric elements 504A). The first and second piezoelectric material layers may then be diced or etched to create a plurality or an array of actuator components 502, one actuator component 5002 per chamber 508 included in the array 600. In some embodiments, the second substrate forming chambers walls 506 is etched or milled only part way therethrough, such that some of the material of the additional substrate 506 remains to form the top surface of each chamber 508. This top surface being situated between the bottom of the actuator components 502 and the chambers 508. Thereafter, an additional set of electrical leads 518B are formed over the second piezoelectric material layer to provide a further electrical contact to each of actuator components 502. Prior to the formation of the additional set of electrical leads, a passivation, isolation, and/or insulation layer may be formed over the second piezoelectric material layer to provide electrical contact at a specific, limited location on the second piezoelectric material layer.

FIG. 7 is perspective diagram of a fluid delivery sub-assembly 700 depicted in its flat form in which it is assembled prior to forming the device into its final form. Embodiments of the fluid delivery sub-assembly 700 may be suitable in the fluid delivery component 110 depicted in FIG. 1. The fluid delivery sub-assembly 700 comprises a flex circuit 702, to which the other illustrated components of the sub-assembly 700 are attached. As depicted, the flex circuit 702 comprises a flexible polyimide film layer (substrate) such as KAPTON™ by DuPont. However, other suit-able flexible and relatively strong materials, such as MYLAR®, also by DuPont, may comprise the substrate of the flex circuit 702. In the depicted embodiment, the flex circuit 702 further comprises metallic interconnection circuitry formed from a malleable metal (such as gold) deposited by means of sputtering, plating and etching techniques employed in the fabrication of microelectronic circuits upon a chromium adhesion layer on a surface of the flex circuit 702.

The interconnection circuitry comprises conductor lines deposited upon the surface of the flex circuit 702 between a set of integrated circuit chips 704 and a set of actuatable chambers 706. In the depicted embodiment, chambers 704 are substantially similar to the chambers 508 of FIGS. 5 and 6. The substrate of flex circuit 702 may be the substrate 510 for the array of chambers 706. The interconnection circuitry also comprises conductor lines between adjacent ones of the integrated circuit chips 704 and a set of cable pads 708 for communicatively coupling the chambers 706 to a controller or sub-controller. The controller may communicate with the set of integrated circuit chips 704 in order to select one or more of the chambers 706 to actuate. In some embodiments, including the depicted embodiment, integrated circuit chips 704 collectively form a multiplexer that allows a controller to select one of the chambers 706 for actuation.

The width “W” of the individual conductor lines of the metallic circuitry (on the order of one-thousandth of an inch) is relatively thin in comparison to the typical width of metallic circuitry deposited upon a film or other flexible substrate. On the other hand, the width of the individual conductor lines is relatively large in comparison to the width of transmission lines in a typical integrated circuit. The layer thickness “T” of the conductor lines between the integrated circuit chips 704 and the array of chambers 706 is preferably 2-5 microns. This selected magnitude for the thickness and the width of the conductor lines enables the conductor lines to be sufficiently conductive while maintaining relative flexibility and resiliency so that the conductor lines do not break during re-shaping of the flex circuit 702. For example, the conductor lines do not break during a re-shaping of the flex circuit 702 into a cylindrical shape.

In the depicted embodiment, the thickness of the substrate of flex circuit 702 is from about 12.5 microns to about 25.0 microns. However, the thickness of the substrate is generally related to the degree of curvature in the final assembled fluid delivery component and may be thinner or thicker accordingly. The thin substrate of the flex circuit 702, as well as the relative flexibility of the substrate material, enables the flex circuit 702 to be wrapped into a generally cylindrical shape after the integrated circuit chips 704 and the array of chambers 706 have been mounted and/or formed and then attached to the metallic conductors of the flex circuit 702. Thus, a flexible substrate thickness may be on the order of several microns to well over 100 microns or more depending upon the flexibility requirements of the particular fluid delivery assembly configuration.

The flex circuit 702 is typically formed into a cylindrical shape in order to accommodate the space limitations of blood vessels in which an intravascular device containing the fluid delivery sub-assembly 700 may be inserted for treatment or through which the fluid delivery sub-assembly may pass en route to a target tissue site. In such instances the range of diameters for the cylindrically shaped fluid delivery component assembly is typically within the range of 0.5 millimeters to 3.0 millimeters. However, it is contemplated that the diameter of the fluid delivery component, when cylindrical, may be on the order of 0.3 millimeters to 5 millimeters. Furthermore, the fluid delivery sub-assembly 700 may also be incorporated into larger cylindrical assemblies, such as may be used on a distal end of flexible elongate member of an intravascular device and provide additional functionality.

FIG. 8 depicts a cross-sectional view of fluid delivery sub-assembly 700 when formed into a cylindrical shape. FIG. 8 depicts a cross-section taken along the array of chambers 706. In the embodiment depicted, two sides of drug-delivery sub-assembly 700 have been joined together to form a cylinder. In other embodiments, the drug-delivery sub-assembly 700 may be positioned within a pre-formed cylinder made from a bio-compatible polymer or metal. As depicted, chambers 706 are positioned inside the full circumference of the cylinder. However, in some embodiments, chambers 706 may be positioned only part way around the circumference of the cylinder. The diameter of the cylindrical shape is generally of an appropriate size for using within the vasculature of a patient. For example, the diameter of the cylindrical may be around 5 millimeters in diameter or less. In certain embodiments, the diameter may be less than 3 millimeters or less than 1 millimeter. In a smaller embodiment, the diameter may be in a range of about 0.3 millimeters to about 0.5 millimeters.

After the fluid delivery sub-assembly 700 is formed into a cylinder, a cylindrical insert 802 is inserted into the cylinder formed by fluid delivery sub-assembly 700. The cylindrical insert 802 may serve multiple purposes. For instance, a pass-through 804 may be formed by the insertion of cylindrical insert 802 that allows electrical lines or support structures, like exemplary structure 805, to pass from one side of the fluid delivery sub-assembly 700 to the other. In embodiments of intravascular devices including fluid delivery sub-assembly 700 in addition to an imaging component situated at a distal tip of the intravascular device, communication and power supply lines may run through the pass through 804. Cylindrical insert 802 may also be used in providing a reservoir 806, by serving as a fluid retaining wall, that can be filled with therapeutic fluid. Reservoir 806 includes a space in between the cylindrical insert 802 and a top surface associated with the array of chambers 706. In some embodiments, a capping layer is formed over the fluid delivery sub-assembly 700 prior to re-shaping.

As discussed above in connection with FIGS. 5 and 6, the reservoir 806 is coupled to each of the array of chambers 706 by a separate inlet with an associated check valve to prevent back flow from the chamber into the reservoir 806. After one chamber of the array of chambers 706 has been actuated, therapeutic fluid contained within reservoir 806 flows through the inlet replenishing the actuated chamber in preparation for additional actuation. In embodiments in which the fluid delivery sub-assembly 700 is inserted into a cylinder, the cylinder may have a plurality of holes therethrough that correspond to outlets associated with each of the array of chambers 706.

FIG. 9 is an additional cross-sectional view of sub-assembly 700 after being formed into a cylinder. FIG. 9 depicts two chambers of the array of chamber 706, including a chamber 902 and a chamber 904, each chamber having a corresponding piezoelectric actuator component 906 and 908, respectively, and an outlet 910 and 912, respectively, that passes through the flexible circuit substrate 702. FIG. 9 also depicts the cylindrical inserting 802 and the reservoir 806 filled with therapeutic fluid.

FIG. 10 is an illustration of an intravascular device 1002 including a fluid delivery component 1004 in a distal portion thereof according to an embodiment. Intravascular device 1102 is similar in many respects to intravascular device 100 of FIG. 1. The intravascular device 1002 is inserted into a vessel 1006. As depicted, vessel 1006 appears to be a blood vessel. However, in some embodiments, the vessel 1006 may be a chamber, such as a cardiac chamber, or a lumen within an organ. Within the vessel 1006, there is an amount of target tissue 1008, which may be cardiac tissue, that is to be treated with portions of a therapeutic fluid. In the depicted embodiment, the fluid delivery component 1004 is an axially directed fluid delivery component, like those depicted in FIGS. 2A and 2B, ejecting portions of the therapeutic fluid parallel to a central axis of the intravascular device 1002.

As depicted, a first portion 1010A is within the target tissue 1008. The volume or dosage of the therapeutic fluid and/or the velocity with which the portion is ejected from the fluid delivery component 1004 may be controlled by an amount of activation energy supplied to the piezoelectric actuator components or component within the fluid delivery component 1004. A second portion 1010B is depicted en route, as ejected, to the location occupied by the droplet or portion 1010A. While the end of the nozzle is shown spaced from the tissue for the purpose of illustration, it will be appreciated that in more preferred application, the nozzle will be in contact with the tissue to inhibit the blood from mixing with fluid being injected. Alternatively, a lower activation energy may be supplied to eject portion 1010B from the fluid delivery component 1004, such that portion 1010B does not penetrate the target tissue 1008 to the same depth reached by portion 1010A. Additionally, by controlling the activation energy, a droplet like portions 1010A and 1010B may be injected through one type of tissue or material and into another. For example, by controlling activating energy and/or a separation distance between the intravascular device 1002 and a target site, a droplet of therapeutic fluid may be injected along path 1012, through tissue 1008, and into the wall of vessel 1006 at target site 1014. Since only the fluid penetrates the tissue or tissues, as compared to a needle-based injection system, the catheter is self-retaining and can deliver repeated drug injections without the need for anchoring to the wall of vessel 1006.

FIG. 11 is an illustration of an intravascular device 1102 that includes a fluid delivery component 1104 in a distal portion thereof according to an embodiment. Thus, intravascular device 1102 is similar to intravascular device 100 of FIG. 1. The fluid delivery component 1104 is similar in many respects to the fluid delivery component 700 depicted in FIGS. 7, 8, and 9, but in some embodiments a single chambered fluid delivery component, like fluid delivery component 500 of FIG. 5, is used. For example, the fluid delivery component 1104 includes an array of actuatable chambers, each of the chambers containing an amount of the therapeutic fluid and configured to eject fluid orthogonally to a central axis of the flexible elongate member of intravascular device 1102. As depicted, the actuatable chambers of fluid delivery component 1104 may form a ring with each of the actuatable chambers configured to eject one or more portions of the therapeutic fluid out a way from the center of the ring. In some embodiments, the fluid delivery component 1104 is structurally coupled to the intravascular device 1102 such that an outer wall of the fluid delivery component 1104 is contiguous with a flexible elongate member of the intravascular device 1102. In other embodiments, the fluid delivery component 1104 is positioned within the flexible elongate member of the intravascular device 1102 such that it is aligned with a plurality of holes extending through the thickness of the flexible elongate member and aligned with each outlet of the array of actuatable chambers.

In the depicted embodiment, the intravascular device 1102 further includes an imaging component 1106 that may be used to image the interior surface of a vessel 1108 within a patient. In this example, the imaging component 1106 is used to locate and identify an inflamed or otherwise damaged target tissue 1110. In one example, the imaging component 1106 can be a phased array intravascular ultrasound (IVUS) assembly. Through the use of a controller, like controller 104 of FIG. 1, coupled to the flexible elongate member of intravascular device 1102, a physician may selectively trigger the actuatable chamber of the array that is closest to the target tissue 1110. To select the particular actuatable chamber, the controller may send an electrical signal to a component controller or sub-controller within the fluid delivery component 1104, such as the multiplexer provided by integrated circuit chips 704 seen in FIG. 7, that is communicatively coupled to that particular actuatable chamber. The physician may use the imaging data obtained by the imaging component 1106 to determine an orientation and position of the intravascular device 1102 relative to the target tissue in order to determine which of the array of actuatable chambers to select. If a better position is obtainable, the physician may use the controller to steer the distal portion of the intravascular device 1102 to that position. Upon determination of the best chamber for use, a piezoelectric actuator component or components may cause the chamber to a contract thereby ejecting a portion 1112 of the therapeutic fluid with sufficient force to penetrate the target tissue 1110. As discussed above with respect to some embodiments, the piezoelectric actuator component or components may cause the chamber to contract when an activation energy is removed rather than when an activation energy is asserted.

FIG. 12 is a flowchart of a method 1200 for applying a therapeutic fluid to a tissue within a patient using an intravascular device or other therapeutic fluid delivery device, such as the devices depicted in FIGS. 1, 10, and 11 as discussed above. Method 1200 includes a plurality of enumerated steps as depicted, but embodiments of method 1200 may include additional steps before, in between, and/or after the enumerated steps. Method 1200 begins with step 1202 in which a physician inserts a therapeutic fluid delivery device into a patient. In step 1204, the physician and/or a computer program positions a distal end of the delivery device close to a target tissue. A number of techniques may be used to determine the proximity of the device to the target tissue. In step 1206, a portion of a therapeutic fluid is ejected with sufficient velocity to contact the tissue without significant elution when an ejection system is actuated. Ejecting the therapeutic fluid may be performed as part of a medical treatment to improve a condition at the site of the target tissue or within the target tissue itself. Ejecting the therapeutic fluid may be performed by actuating at least one piezoelectric actuator component. After the treatment is completed, the physician removes the delivery device from the patient in step 1208. Embodiments of intravascular devices that may be used in performing method 1200 are depicted in FIGS. 1, 10, and 11 and described throughout this disclosure.

In some embodiments, in addition to a fluid delivery component included in the intravascular device, there is also an imaging component. The imaging component, like the fluid delivery component, is positioned in a distal portion of the flexible elongate member of the intravascular device. The imaging component may be used to visualize at least a portion of the surroundings of the distal portion of the flexible elongate member of the intravascular device. The imaging component, which may be an intravascular ultrasound component, may be used to locate and to identify a site within the patient requiring treatment. In some instances of a diagnosis may be made using the imaging component. A particular tissue or portion of tissue within the patient may be chosen as the target tissue or target site in which to inject an amount of the therapeutic fluid. After the target tissue has been identified, in embodiments in which the fluid delivery component includes a plurality of piezoelectrically actuatable chambers, is specific one of the piezoelectrically actuatable chambers may be selected for actuation.

To actuate the chamber, an activation energy may be applied to a piezoelectric actuator component in communication with chamber such that the piezoelectric actuator component physically reacts to alter a volume of the chamber. The alteration of the volume of the chamber is used to apply a force to the therapeutic fluid therein to eject a portion of the therapeutic fluid from the chamber through an outlet. In some embodiments, the portion of therapeutic fluid ejected by a single actuated event may range in volume from about a microliter to about a picoliter. The force provided by the piezoelectrically actuated component may eject the portion of the therapeutic fluid with a velocity of about 10 to about 25 meters per second, which may be sufficient to penetrate the target tissue. Both the volume of the portion of therapeutic fluid and or the ejection velocity may be determined by an amount of activation energy applied to the piezoelectric actuator.

Persons skilled in the art will also recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. An flexible therapeutic fluid delivery device comprising:
a flexible elongate member having a proximal portion and a distal portion, the flexible elongate member configured for minimally invasive insertion into a patient,
a chamber situated in the distal portion of the flexible elongate member, the chamber having an outlet configured to eject a therapeutic fluid;
a piezoelectric actuator positioned adjacent to the chamber such that, when actuated, the piezoelectric actuator alters a volume of the chamber to eject a portion of the therapeutic fluid; and
a plurality of electrical leads coupled to the piezoelectric actuator.

2. The device of claim 1, further comprising an inlet to the chamber, wherein the inlet couples the chamber to a reservoir also containing the therapeutic fluid, the inlet comprising a check valve to prevent fluid from flowing from the chamber into the reservoir while allowing fluid to flow from the reservoir into the chamber.

3. The device of claim 1, wherein the device is a steerable intravascular device.

4. The device of claim 1, wherein the outlet is comprises a tapered section.

5. The device of claim 1, wherein the outlet is coupled to a housing provided at the distal end of the flexible elongate member.

6. The device of claim 1, wherein the outlet and a piezoelectric actuator element are configured on opposite sides of the chamber.

7. The device of claim 1, wherein the piezoelectric actuator comprises a pair of piezoelectric actuator elements configured opposite each other, along opposing walls of the chamber.

8. The device of claim 1, wherein the piezoelectric actuator increases the volume of the chamber when an activation energy is applied.

9. The device of claim 1, wherein the piezoelectric actuator decreases the volume of the chamber when an activation energy is applied.

10. The device of claim 1, wherein the piezoelectric actuator ejects the portion of the therapeutic fluid at a velocity sufficient to inject the portion into tissue adjacent to the outlet.

11. The device of claim 1, wherein the piezoelectric actuator ejects the portion of the therapeutic fluid at a velocity greater than 10 meters per second.

12. The device of claim 1, wherein the piezoelectric actuator ejects the portion of the therapeutic fluid at a velocity greater than 20 meters per second.

13. The device of claim 1, wherein the piezoelectric actuator ejects the portion of the therapeutic fluid at a velocity about 25 meters per second.

14. The device of claim 1, wherein the portion of therapeutic fluid ejected from the chamber has a volume of less than about 5 picoliters.

15. The device of claim 1, wherein the portion of therapeutic fluid ejected from the chamber has a volume of less than about 25 picoliters.

16. The device of claim 1, wherein the portion of therapeutic fluid ejected from the chamber has a volume in a range from about a microliter to about a picoliter.

17. The device of claim 1, wherein the outlet is oriented parallel to a central axis of the flexible elongate member.

18. The device of claim 1, wherein the outlet is oriented perpendicular to a central axis of the flexible elongate member.

19. The device of claim 1, wherein the therapeutic fluid comprises a drug.

20. The device of claim 1, wherein the therapeutic fluid comprises cells.

21. The device of claim 1, further comprising an imaging component positioned in the distal portion of the flexible elongate member.

22. A method for applying a therapeutic fluid to a tissue within a patient using an elongated fluid delivery device, the method comprising:
inserting the elongated fluid delivery device into a patient, the elongated fluid delivery device having an electrically actuated ejection system adjacent a distal end thereof;
positioning the distal end of the elongated fluid delivery device close to the tissue;
actuating the ejection system to eject a portion of a therapeutic fluid with sufficient velocity to contact the tissue; and
removing the elongated fluid delivery device from the patient.

23. The method of claim 22, wherein actuating the ejection system comprises using at least one piezoelectric actuator.

24. The method of claim 22, wherein the sufficient velocity to contact the tissue is sufficient velocity to penetrate the tissue.

25. The method of claim 22, wherein positioning the device close to the tissue comprises determining a site on the tissue at which to apply the therapeutic fluid using an imaging component.

26. The method of claim 25, wherein the imaging component is an intravascular ultrasound component disposed on the fluid delivery device.

27. The method of claim 22, further comprising priming a chamber containing the therapeutic fluid.

28. The method of claim 22, wherein the portion of therapeutic fluid of a volume from about a microliter to about a picoliter.

29. The method of claim 22, wherein a volume of the portion of the therapeutic fluid is determined by an amount of the activation energy applied to a piezoelectric actuator.

30. The method of claim 22, wherein the tissue is cardiac tissue.
